(19) United States
(12) Patent Application Publication
(10) Pub. No.: US 2010/0312251 A1
(43) Pub. Date: Dec. 9, 2010

(54) Pedicle Driver

(76) Inventor: Fraser Landreneau, Baton Rouge, LA (US)

Correspondence Address: nolaIP, LLC, P.O. BOX 24100, NEW ORLEANS, LA, 70184, US

(21) Appl. No.: 12/792,749

(22) Filed: Jun. 3, 2010

Related U.S. Application Data
(60) Provisional application No. 61/183,628, filed on Jun. 3, 2009.

(51) Int. Cl. A61B 17/56

(52) U.S. Cl. 606/104

(57) ABSTRACT

An improved detachable pedicle driver is disclosed having various external features to facilitate gauging depth by a surgeon, as well as being easier to manipulate by virtue of its revised side attachment design and light weight.

### CONTINUATION HISTORY

This application is a continuation of U.S. Patent Application 61/183,628, filed on Jun. 3, 2009, which is hereby incorporated by reference in its entirety.

### BACKGROUND OF THE INVENTION

The spine is one of the most critical components of the human body, and serves many purposes, both structural and neural. Structurally, the spine supports the back, and connects the upper and lower skeletal systems. The neural portion, commonly known as the spinal cord, is composed of various nerves which exit the spinal cord at various points throughout its length. In normal cases, these nerves are protected by the spine, a relatively rigid component, against damage.

In order for this rigid spine to provide protection as well as mobility for the human body, the spine's curvature is comprised of several vertebrae separated by intervertebral discs. These discs permit movement of the vertebrae relative to one another in both horizontal and vertical axes. In an unhealthy or damaged spine, these discs can become herniated, broken or otherwise damaged. The disc height is thusly reduced, reducing the spacing between these vertebrae, which exerts pressure on these nerves, in turn causing damage to the nerves, and often, severe pain.

To repair these damaged spinal components and alleviate the symptoms caused by same, several methods may be employed in surgical procedures. Many times, components, such as grafts, screws, rods, cages or the like may be inserted in the disc space to restore the proper height and relieve pressure on the nerves. These various components are most often held in place by anchors, one of the most frequently used of which is commonly known as a pedicle screw. These pedicle screws come in various different applications for different inserted components, spinal sizes and so forth.

Various tools have been employed to place and secure these pedicle screws, and an improved tool for driving these screws is disclosed as the subject of this invention.

### OBJECTS OF THE INVENTION

An object of this invention is to provide an apparatus for driving pedicle screws which facilitates gauging depth without the use of x-ray.

Another object of this invention is to provide an apparatus for driving pedicle screws which has graduations to indicate the depth of the pedicle screw being driven by a surgeon.

Yet another object of this invention is to provide an apparatus for driving pedicle screws which is detachable on the shaft portion of the driver,

Still another object of this invention is to provide an apparatus for driving pedicle screws which includes radiopaque graduations.

Other objects and advantages of this invention shall become apparent from the ensuing descriptions of the invention.

### SUMMARY OF THE INVENTION

According to the present invention, an improved detachable pedicle driver is disclosed having various external features to facilitate gauging depth by a surgeon, as well as being easier to manipulate by virtue of its revised side attachment design and light weight.

### BRIEF DESCRIPTION OF THE DRAWINGS

The accompanying drawings illustrate an embodiment of this invention, However, it is to be understood that this embodiment is intended to be neither exhaustive, nor limiting of the invention. They are but examples of some of the forms in which the invention may be practiced.

FIG. 1 displays a first side view of the driver and detachable driver portions of the tool.

FIG. 2 displays a second side view of the driver and detachable driver portions of the tool.

FIG. 3 displays a third side view of the driver and detachable driver portions of the tool.

FIG. 4 displays a first side view of a detachable driver,

FIG. 5 displays a second side view of a detachable driver,

FIG. 6 displays a third side view of a detachable driver.

### DETAILED DESCRIPTION OF EXEMPLARY EMBODIMENTS

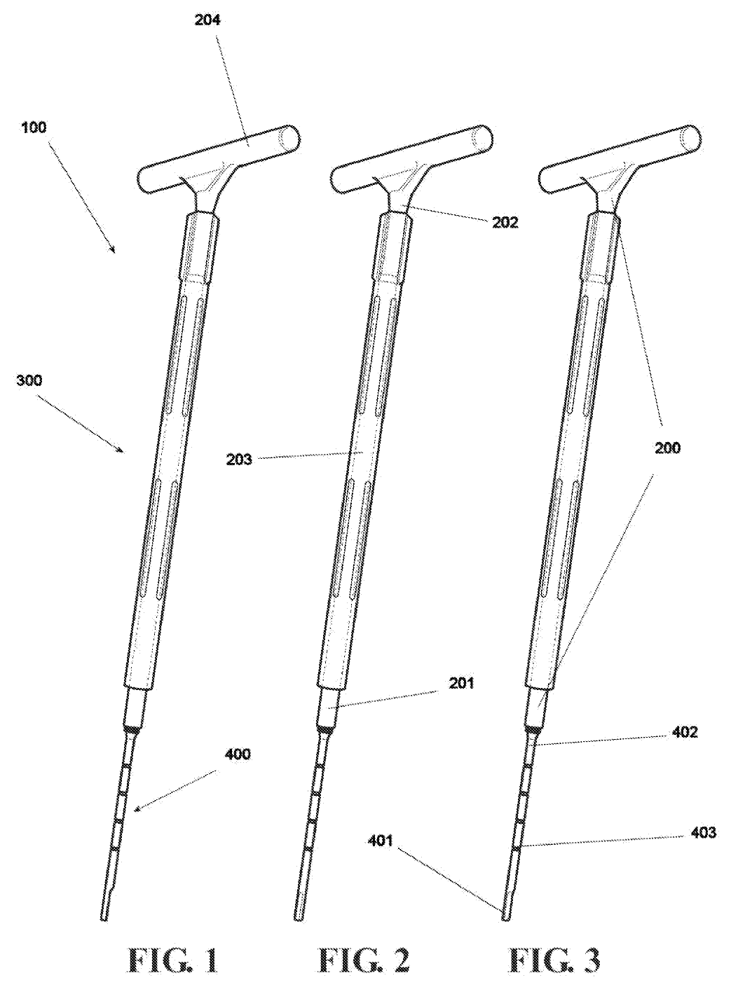

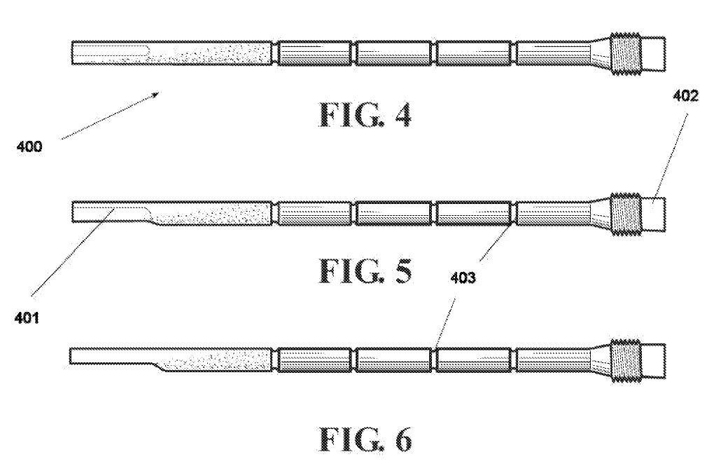

Without any intent to limit the scope of this invention, reference is made to the figures in describing the various embodiments of the invention. FIGS. 1-6 show various aspects of exemplary embodiments of the present invention,

The present invention is a pedicle screw driver 100. Three main components comprise the pedicle screw driver 100: drive member 200, sleeve member 300 and driver 400.

Drive member 200 has first opposite end 201 which is a portion of the tool configured to attach to driver 400 in an easily detachable arrangement. This configuration may be by having a keyed relationship, threads, or any other longitudinal attachment mechanism. This detachable relationship is advantageous over existing tools because drive member 200 can be detached from driver 400 to permit the user of the device to leave driver 400 in the patient to check alignment, depth and other attributes of the pedicle screw without the tool 100 remaining in the way of the user. In this fashion, the surgeon can perform more manipulations, etc. before the pedicle screw is finally set, increasing precision and ease of use. This has the added benefit of making the task of driving the screw easier for the surgeon, reducing fatigue and the time necessary to drive the pedicle screw. Moreover, a more precise application of the pedicle screw leads to improved surgical results, shorter recovery times and reduced errors.

Drive member 200 also has second opposite end 202 which is a handle 204, either molded as part of drive member 200 or otherwise attached to second opposite end 202. A longitudinal axis 203 exists between first opposite end 201 and second opposite end 202 of drive member 200. Handle 204 is, in the exemplary embodiment as seen in FIGS. 1-3 substantially perpendicular to longitudinal axis 203. This configuration permits a more friendly application to the user, increasing the torque available to the user in driving drive member 200 and reducing the dexterity required to operate the tool 100.

Sleeve member 300 is designed to engage the exterior of drive member 200, forming a coaxial relationship between sleeve member 300 and drive member 200, whereby drive member 200 may be slid in and out of sleeve member 300. The interior of sleeve member 300 is designed to have a portion therein which is keyed, or shaped in a way to correspond to a matching keyed portion of drive member 200 such that the two can either be operated independently in certain sliding positions, or the two keyed portions engaged such that drive member 200 and sleeve member 300 operate in a dependent fashion on other sliding positions. This facilitates the torquing of the drive member 200 either independently or dependently with the sleeve member 300.

Drivers 400 are configured to have a first opposite end 401 and second opposite end 402. First opposite end 401 is designed to engage a pedicle screw or any other device that may be desired to be driven. Second opposite end 402 is configured to attach to drive member 200's first opposite end 201. Driver 400 can be configured, as in the pictured exemplary embodiment in FIGS. 4-6 to have graduations 403 along the exterior so that insertion depth can be readily gauged, This is critical so that the user or surgeon can determine what depth the pedicle screw has been driven thus far into the spine. Driver 400 can also be configured to have a radiopaque exterior so that it can be visible easily under x-ray. Shading or coloring can also be employed, by putting one color at one stage or depth of driver 400 and another color at other points along the driver 400. Having this color coding enables a user to very quickly ascertain depth and can operate independently or jointly with a radiopaque exterior and/or graduations 403.

In all cases, drive member 200, sleeve member 300 and driver 400 are to be manufactured of lightweight material to reduce operator fatigue and aid in the surgical procedure. This arrangement of components and side attachment design is configured in lieu of the commonly used end-on-end designs of other tools

Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention as defined in the following claims.

The invention claimed is:

1. A pedicle screw driver for inserting a pedicle screw comprising:
a. a drive member having first and second opposite ends and a longitudinal axis between; said first end being configured to attach to a driver, said second end being configured to form a handle substantially perpendicular to said longitudinal axis;
b. a sleeve member operatively configured to surround a substantial portion of said longitudinal axis of said drive member, and which defines said drive member and said sleeve in a coaxial relationship;
c. said sleeve member further configured to have a keyed portion along an inner wall of said sleeve member;
d. said drive member further configured to have a corresponding keyed portion configured to operatively engage said sleeve member's keyed portion; and
e. at least one driver having first and second opposite ends, wherein said first opposite end is configured to drive a pedicle screw, and wherein said second opposite end is configured to attach to said first end of said drive member.

2. The pedicle screw driver for inserting a pedicle screw of claim 1 wherein said driver is further comprises graduations interspersed along the exterior of said driver.

3. The pedicle screw driver for inserting a pedicle screw of claim 1 wherein the exterior of said driver is further configured to be radiopaque.

4. The pedicle screw driver for inserting a pedicle screw of claim 1 wherein the exterior of said driver is further configured to be color coded based on depth.

5. The pedicle screw driver for inserting a pedicle screw of claim 1 wherein said driver is comprised of a lightweight material.

6. A pedicle screw driver for inserting a pedicle screw comprising:
a. a drive member having first and second opposite ends and a longitudinal axis between; said first end being configured to attach to a driver, said second end being configured to form a handle substantially perpendicular to said longitudinal axis;
b. a sleeve member operatively configured to surround a substantial portion of said longitudinal axis of said drive member, and which defines said drive member and said sleeve in a coaxial relationship;
c. said sleeve member further configured to have a keyed portion along an inner wall of said sleeve member;
d. said drive member further configured to have a corresponding keyed portion configured to operatively engage said sleeve member's keyed portion;
e. at least one driver having first and second opposite ends, wherein said first opposite end is configured to drive a pedicle screw, and wherein said second opposite end is configured to attach to said first end of said drive member;
f. graduations interspersed along the exterior of said driver;
g. a radiopaque coating on the exterior of said driver; and
h. depth-based color-coding applied to the exterior of said driver.
